(19) United States
(12) Patent Application Publication
(10) Pub. No.: US 2021/0256830 A1
(43) Pub. Date: Aug. 19, 2021

(54) Wall Power Supply with Gas Detection

(71) Applicant: Sherrie Lynn Liebenow, Treasure Island, FL (US)

(72) Inventor: Sherrie Lynn Liebenow, Treasure Island, FL (US)

(21) Appl. No.: 16/792,196

(22) Filed: Feb. 15, 2020

(51) Int. Cl. G08B 21/14; G08B 25/10; G01N 33/00

(52) U.S. Cl. CPC G08B 21/14; G01N 33/004; G08B 25/10

(57) ABSTRACT

A system for detecting a concentration of carbon monoxide includes a wall transformer/power-supply having a membrane for passing of the carbon monoxide from outside of the wall transformer/power-supply to inside the wall transformer/power-supply. A sensor within the wall transformer/power-supply is coupled to the membrane for receiving the carbon monoxide. The sensor provides an electrical signal proportional to the concentration of the carbon monoxide. A circuit within the wall transformer/power-supply receives the electrical signal and compares the electrical signal to a concentration threshold and the circuit alerts if the electrical signal exceeds the concentration threshold. A power conversion circuit within the wall transformer/power-supply accepts a household power and providing power to a device connected to the wall transformer/power-supply.

### FIELD

This invention relates to the field of power supplies and more particularly to a wall power-supply having smoke/gas detection.

### BACKGROUND

Many people die each year from fires and/or carbon monoxide poisoning. Carbon monoxide (CO) is a colorless, odorless gas that readily diffuses into air, substantially evenly at all layers of air within a room. Therefore, a carbon monoxide detector will operate properly at floor levels, at power outlet levels, at countertop levels, etc.

Many homes and some vehicles (e.g. boats) are equipped with carbon monoxide detectors, especially when fossil fuel is burned for heat, movement, or generation of electricity. These carbon monoxide detectors are relatively inexpensive and some are portable. For example, one such carbon monoxide detector operates from two AA batteries and weighs eight ounces (without batteries), measuring 3″ by 7″ by 11″. Such devices are great for home use, but often people travel and stay in hotels, cabins, bed-and-breakfast rooms, etc. Some such travel destinations accommodate the safety of those staying, but many do not or many do not properly maintain the devices, leading to unsafe conditions. Several deaths each year result from carbon monoxide poisoning in such travel destinations.

Many travelers would be safer if there is a carbon monoxide (and/or smoke) detector in whatever rooms they stay, but as stated above, there is no guarantee of a carbon monoxide detector. This leaves many travelers who are concerned with their well-being to pack a carbon monoxide and/or smoke detector in their luggage when they travel. Unfortunately, it is easy to forget to take the carbon monoxide detector as it is not an everyday use item.

Many people now have cellphones and are very accustomed to charging their cellphones, typically using a wall transformer or wall power-supply (a transformer or power-supply that plugs directly into a wall outlet). Knowing that their cellphone will only last hours without being recharged, it is rare for a cellphone user to forget their wall transformer/power-supply when traveling.

What is needed is a wall transformer/supply with an integrated gas/smoke detector.

### SUMMARY

In one embodiment, a system for detecting a concentration of a gas is disclosed including a wall transformer/power-supply having a membrane for passing of the gas from outside of the wall transformer/power-supply to inside the wall transformer/power-supply. A sensor within the wall transformer/power-supply is coupled to the membrane for receiving the gas. The sensor provides an electrical signal proportional to the concentration of the gas. A circuit within the wall transformer/power-supply receives the electrical signal and compares the electrical signal to a concentration threshold and the circuit alerts if the electrical signal exceeds the concentration threshold. A power conversion circuit within the wall transformer/power-supply accepts a household power and provides power to a device connected to the wall transformer/power-supply.

In another embodiment, a method of detecting a gas is disclosed including receiving external power into a wall transformer/power-supply and converting the external power into a direct current power for powering a device. Receiving the gas through a membrane in a wall transformer/power-supply and detecting a concentration of the gas by a circuit within the wall transformer/power-supply. An alarm is signaled if the concentration is greater than a preset threshold.

In another embodiment, a system for detecting a concentration of carbon monoxide is disclosed including a wall transformer/power-supply having a membrane for passing of the carbon monoxide from outside of the wall transformer/power-supply to inside the wall transformer/power-supply. A sensor within the wall transformer/power-supply is coupled to the membrane for receiving the carbon monoxide. The sensor provides an electrical signal proportional to the concentration of the carbon monoxide. A circuit within the wall transformer/power-supply receives the electrical signal and compares the electrical signal to a concentration threshold and the circuit alerts if the electrical signal exceeds the concentration threshold. A power conversion circuit within the wall transformer/power-supply accepts a household power and providing power to a device connected to the wall transformer/power-supply.

### BRIEF DESCRIPTION OF THE DRAWINGS

The invention can be best understood by those having ordinary skill in the art by reference to the following detailed description when considered in conjunction with the accompanying drawings in which:

FIG. 1 illustrates a wall transformer/supply of the prior art.

FIG. 2 illustrates a wall transformer/supply having an integrated gas detector.

FIG. 3 illustrates a schematic view of a typical cellphone.

FIG. 4 illustrates the wall transformer/supply having an integrated gas detector communicating with the cellphone by a wired connection.

FIG. 5 illustrates the wall transformer/supply having an integrated gas detector communicating with the cellphone by a wireless connection.

FIG. 6 illustrates a schematic diagram of the wall transformer/supply having an integrated gas detector and sound emitting device.

FIG. 7 illustrates a schematic diagram of the wall transformer/supply having an integrated gas detector communicating with the cellphone by a wired connection.

FIG. 8 illustrates a schematic diagram of the wall transformer/supply having an integrated gas detector communicating with the cellphone by a wireless connection.

### DETAILED DESCRIPTION

Reference will now be made in detail to the presently preferred embodiments of the invention, examples of which are illustrated in the accompanying drawings. Throughout the following detailed description, the same reference numerals refer to the same elements in all figures.

In general, the wall power-supply with gas detection provides a wall power-supply similar to the prior art, except having an integrated gas detector (e.g. carbon monoxide detector) that detects a presence of the gas (gases) and informs anyone in the proximity that the gas is present by an integrated sound emitting device and/or by signaling a local cellphone and the cellphone alerting nearby people.

Throughout this description, the term, “wall power-supply” refers to any power-supply that is external to an electronic device that is to be powered and interfaces to a household power connection/outlet. Although shown connected to a cellphone in the examples, wall power supplies are known for powering/charging monitors, notebook computers, tablet computers, electronic books, etc., all of which are anticipated herein.

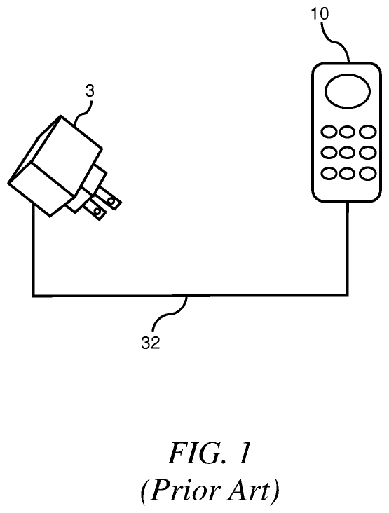

Referring to FIG. 1, a wall transformer/supply 3 of the prior art is shown. In this example of the prior art, a typical wall transformer/supply 3 powers/charges a cellphone 10 through a cable 32, though it is anticipated that, in some embodiments, the connection to the cellphone 10 for charging is wireless. Although a cellphone 10 is shown, wall the transformer/supply 3 is known to be used in powering/charging monitors, notebook computers, tablet computers, electronic books, etc.

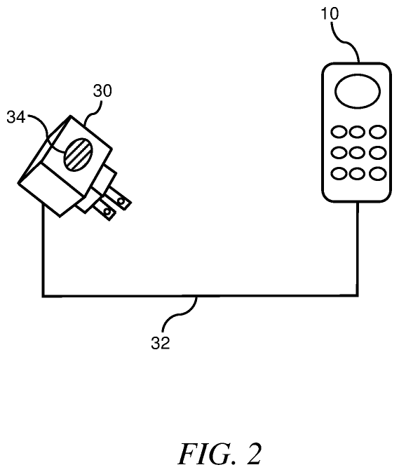

Referring to FIG. 2, a wall transformer/supply 30 having an integrated gas detector is shown. In this example, the wall transformer/supply 30 powers/charges a cellphone 10 through a cable 32, though it is anticipated that, in some embodiments, the connection to the cellphone 10 for charging is wireless. The wall transformer/supply 30 is shown having a gas-permeable membrane 34 through which the gas (e.g. carbon monoxide) will pass so as to be detected by the wall transformer/supply 30 having an integrated gas detector.

Although a cellphone 10 is shown, the wall transformer/supply 30 having an integrated gas detector is known to be used in powering/charging monitors, notebook computers, tablet computers, electronic books, etc., all of which are equally anticipated.

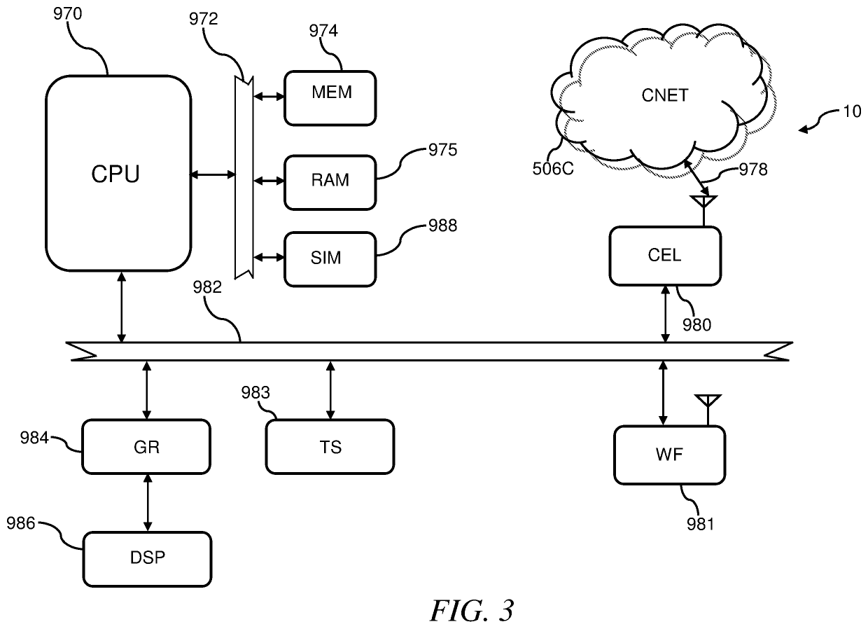

Referring to FIG. 3, a schematic view of an exemplary cellphone 10 is shown. The present invention is in no way limited to any particular cellphone 10 and many other devices are anticipated that require similar charging such as tablet computers, notebook computers, wireless speakers, etc.

The exemplary cellphone 10 represents a typical device that requires periodic charging. This exemplary cellphone 10 is shown in its simplest form. Different architectures are known that accomplish similar results in a similar fashion, and the present invention is not limited in any way to any particular system architecture or implementation. In this exemplary cellphone 10, a processor 970 executes or runs programs in a random access memory 975. The programs are generally stored within a persistent memory 974 and loaded into the random access memory 975 when executed. A subscriber identity module 988 (SIM or SIM card) securely stores an international mobile subscriber identity (IMSI) number and its related key, which are used to identify and authenticate subscribers on the cellular network 506C. The processor 970 is any processor, typically a processor designed for data communications. The persistent memory 974, random access memory 975, and subscriber identity module 988 are connected to the processor by, for example, a memory bus 972. The random access memory 975 is any memory suitable for connection and operation with the selected processor 970, such as SRAM, DRAM, SDRAM, RDRAM, DDR, DDR-2, etc. The persistent memory 974 is any type, configuration, capacity of memory suitable for persistently storing data, for example, flash memory, read only memory, battery-backed memory, etc.

Also connected to the processor 970 is a system bus 982 for connecting to peripheral subsystems such as a cellular network interface 980 (e.g. cellular interface) and a local wireless network interface 981 (e.g. Wi-Fi). A graphics adapter 984 is provided for driving a display 986, and an touch screen input device 983 is provided for accepting user inputs. Note that there is no restriction on inputs and outputs.

In general, some portion of the persistent memory 974 is used to store programs, executable code, and data, etc.

The peripherals are examples, and other devices are known in the industry are anticipated, the details of which are not shown for brevity and clarity reasons.

The cellular network interface 980 connects the cellphone 10 to the cellular network 506C through any known or future protocol such as GSM, TDMA, LTE, etc. There is no limitation on the type of cellular connection used. The cellular network interface 980 provides data and messaging between the exemplary cellphone 10 and the cellular network 506C.

In some embodiments, a local wireless network interface 981 connects the cellphone 10 to a local wireless network through any known or future protocol such as Wi-Fi (802.11x), Bluetooth, etc. There is no limitation on the type of local wireless connection used. The local wireless network interface 981 provides data and messaging between the cellphone 10 and other devices, and in some embodiments, to the gas/smoke detector.

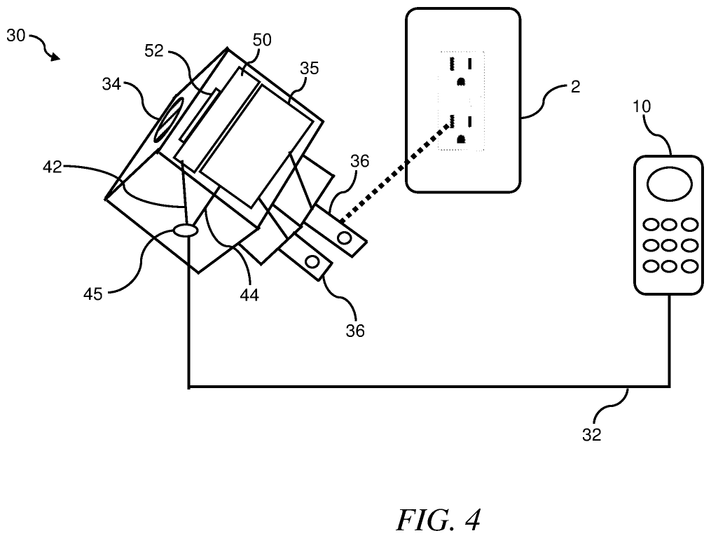

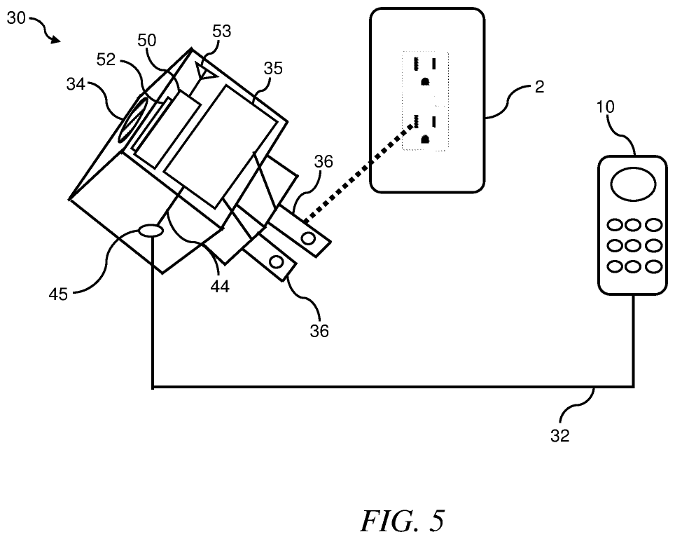

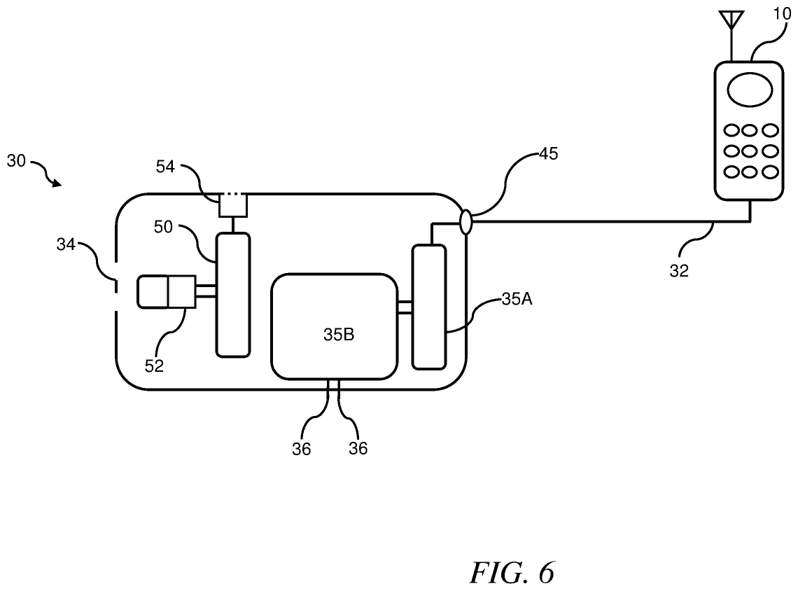

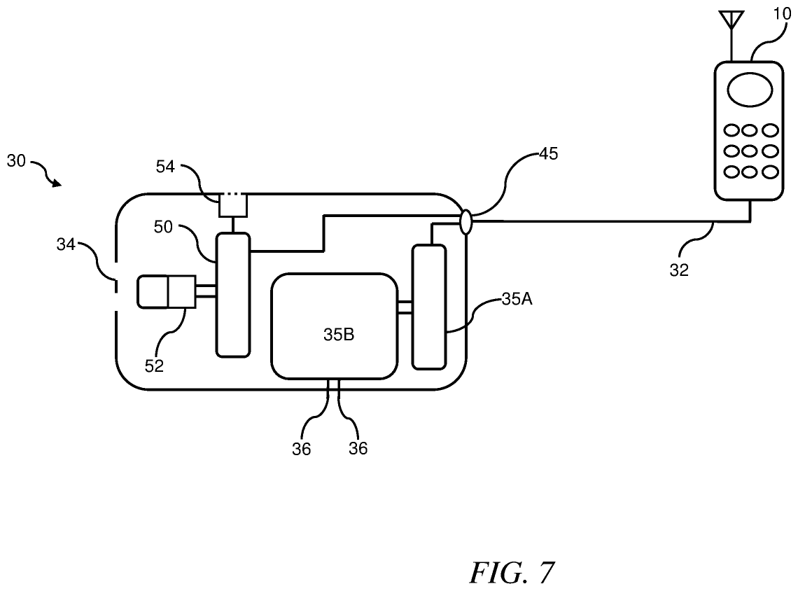

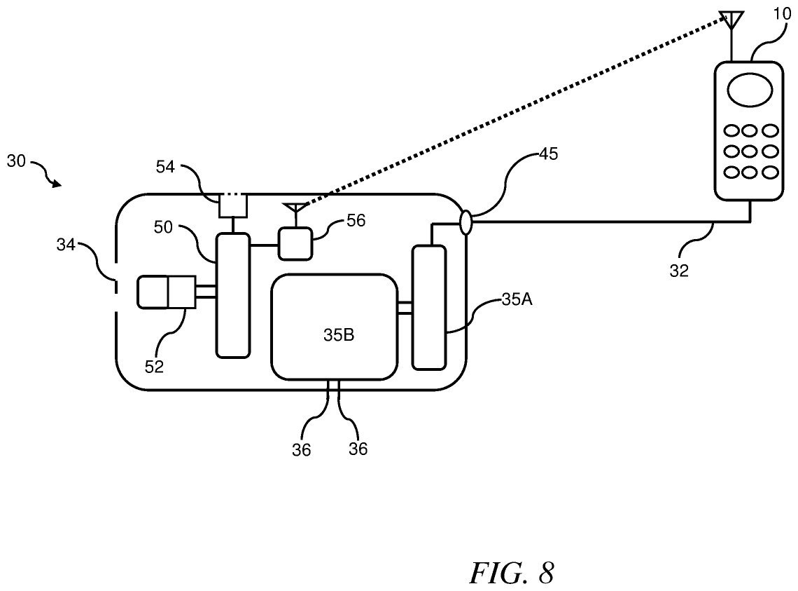

Referring to FIGS. 4-8, the wall transformer/supply 30 having an integrated gas detector is shown. In FIGS. 4 and 7, the wall transformer/supply 30 is shown communicating with the cellphone by a wired connection; in FIGS. 5 and 8, the wall transformer/supply 30 is shown communicating with the cellphone by a wireless connection; and in FIG. 6, the wall transformer/supply 30 is shown having an integrated sound emitting device 54.

In FIG. 4, the prongs 36 of the wall transformer/supply 30 are shown interfacing with a standard power outlet 2. Power from the prongs 36 is connected to a transformer/power-conditioner 35 to provide the proper power (voltage, current, AC or DC) to the intended device (e.g. the cellphone 10). The wall transformer/supply 30 includes the gas-permeable membrane 34 (e.g. screen) that allows the flow of the target gas/gases (e.g. carbon monoxide) into the wall transformer/supply 30 and onto a gas sensor 52 that is connected to internal circuitry 50 that determines when a concentration of the gas/gases reaches a predetermined threshold. In the embodiment of FIG. 4, the internal circuitry 50 is connected 42 to pins of the connector 45 communicates with the device (cellphone 10). Power 44 is also connected to the connector 45 for powering/charging the device (cellphone 10).

In FIG. 5, only power 44 is provided to the connector 45 and the internal circuitry 50 includes a wireless transmitter with antenna 53 (e.g. Bluetooth) which, when paired with the device (cellphone 10), alerts the device (cellphone 10) of when a concentration of the gas/gases reaches a predetermined threshold. In such, the device (cellphone 10) receives the alert and takes action to inform those present near the wall transformer/supply 30 having an integrated gas detector of the impending danger.

In FIG. 6, power from the prongs 36 of the wall transformer/supply 30 is connected to a transformer 35B and the transformer 35B powers a regulator 35A to provide the proper power DC power to the intended device (e.g. the cellphone 10), for example, five volts, direct current. The wall transformer/supply 30 includes the gas-permeable membrane 34 (e.g. screen) that allows the flow of the target gas/gases (e.g. carbon monoxide) into the wall transformer/supply 30 and onto a gas sensor 52 that is connected to internal circuitry 50 that determines when a concentration of the gas/gases reaches a predetermined threshold. For example, the gas sensor 52 converts concentration levels of the gas into an electrical signal and the internal circuitry compares the electrical signal to a predetermined threshold. In the embodiment of FIG. 6, the internal circuitry 50 is connected to a sound emitting device 54 (e.g. piezo sounder, buzzer . . . ). Power from the regulator 35A is connected to the connector 45 for powering/charging the device (cellphone 10).

In FIG. 7, power from the prongs 36 of the wall transformer/supply 30 is connected to a transformer 35B and the transformer 35B powers a regulator 35A to provide the proper power DC power to the intended device (e.g. the cellphone 10), for example, five volts, direct current. The wall transformer/supply 30 includes the gas-permeable membrane 34 (e.g. screen) that allows the flow of the target gas/gases (e.g. carbon monoxide) into the wall transformer/supply 30 and onto a gas sensor 52 that is connected to internal circuitry 50 that determines when a concentration of the gas/gases reaches a predetermined threshold. For example, the gas sensor 52 converts concentration levels of the gas into an electrical signal and the internal circuitry compares the electrical signal to a predetermined threshold. In the embodiment of FIG. 7, the internal circuitry 50 is connected 42 to pins of the connector 45 communicates with the device (cellphone 10). Power from the regulator 35A is also connected to the connector 45 for powering/charging the device (cellphone 10).

In some embodiments, as shown in FIG. 7, the internal circuitry 50 is also optionally connected to a sound emitting device 54 (e.g. piezo sounder, buzzer . . . ).

In FIG. 8, a power conversion circuit takes power from the prongs 36 of the wall transformer/supply 30. This external power is connected to a transformer 35B and the transformer 35B powers a regulator 35A to provide the proper power DC power to the intended device (e.g. the cellphone 10), for example, five volts, direct current. The wall transformer/supply 30 includes the gas-permeable membrane 34 (e.g. screen) that allows the flow of the target gas/gases (e.g. carbon monoxide) into the wall transformer/supply 30 and onto a gas sensor 52 that is connected to internal circuitry 50 that determines when a concentration of the gas/gases reaches a predetermined threshold. For example, the gas sensor 52 converts concentration levels of the gas into an electrical signal and the internal circuitry compares the electrical signal to a predetermined threshold. In the embodiment of FIG. 8, the internal circuitry 50 is connected to a transmitter/transceiver 56 (e.g. Bluetooth transceiver) that communicates (e.g. sends a signal) wirelessly with the device (cellphone 10).

The pre-determined threshold is anticipated to be factory set or adjusted, for example, through the device (e.g. cellphone 10).

Power from the regulator 35A is connected to the connector 45 for powering/charging the device (cellphone 10).

In some embodiments, as shown in FIG. 8, the internal circuitry 50 is also optionally connected to a sound emitting device 54 (e.g. piezo sounder, buzzer . . . ). This is useful when the device (e.g. cellphone 10) is not present and the concentration of the gas/gases is detected.

In some embodiments, software running on the device (e.g. cellphone 10) automatically initiates contact when the concentration of the gas exceeds the threshold. For example, the cellphone 10 sends a Short Message Signaling message to another cellphone 10.

Equivalent elements can be substituted for the ones set forth above such that they perform in substantially the same manner in substantially the same way for achieving substantially the same result.

It is believed that the system and method as described and many of its attendant advantages will be understood by the foregoing description. It is also believed that it will be apparent that various changes may be made in the form, construction and arrangement of the components thereof without departing from the scope and spirit of the invention or without sacrificing all of its material advantages. The form herein before described being merely exemplary and explanatory embodiment thereof. It is the intention of the following claims to encompass and include such changes.

1. A system for detecting a concentration of a gas, the system comprising:
a wall transformer/power-supply having an enclosure;
a membrane for passing of the gas from outside of the enclosure to inside the enclosure;
a sensor within the enclosure wall and coupled to the membrane for receiving the gas, the sensor provides an electrical signal proportional to the concentration of the gas;
a circuit within the enclosure, the circuit receives the electrical signal and compares the electrical signal to a concentration threshold and the circuit alerts if the electrical signal exceeds the concentration threshold;
a pair of electrical contacts passing through the enclosure, the pair of electrical contacts for mechanically and electrically interfacing with a power outlet;
a power conversion circuit within the enclosure is electrically interfaced to the pair of electrical contacts and the power conversion circuit converts power from the power outlet into a different power and provides the different power to a device connected to the wall transformer/power-supply.

2. The system for detecting the concentration of the gas of claim 1, wherein when the circuit alerts, a sound is emitted from a sound emitting device mounted within the enclosure.

3. The system for detecting the concentration of the gas of claim 1, wherein when the circuit alerts, a signal is sent from the circuit to the device connected to the wall transformer/power-supply by a wire and the device emits a sound.

4. The system for detecting the concentration of the gas of claim 3, wherein the device is a cellphone.

5. The system for detecting the concentration of the gas of claim 1, wherein when the circuit alerts, a signal is sent from the circuit to the device by a wireless transceiver and the device emits a sound.

6. The system for detecting the concentration of the gas of claim 5, wherein the device is a cellphone.

7. The system for detecting the concentration of the gas of claim 1, wherein the gas is carbon monoxide.

8. A method of detecting a gas, the method comprising:
inserting into a wall outlet a pair of metal prongs that pass through an enclosure of a wall transformer/power supply;
electrically connecting the wall transformer/power supply to a device;
within the wall transformer/power supply, receiving electrical power from the pair of metal prongs and converting the electrical power into a direct current power powering the device;
receiving the gas through a membrane in a surface of the enclosure;
detecting a concentration of the gas by a circuit within the enclosure;
if the concentration is greater than a preset threshold, signaling an alarm.

9. The method of claim 8, wherein the signaling of the alarm comprises emitting a sound from a sound emitting device of the wall transformer/power-supply.

10. The method of claim 8, wherein the signaling of the alarm comprises sending of a message to the device and, upon receiving the message, the device emitting at least a sound.

11. The method of claim 8, wherein the sending of the message to the device is performed wirelessly.

12. The method of claim 11, wherein the sending of the message to the device is performed wirelessly using Bluetooth®.

13. The method of claim 8, wherein the gas is carbon monoxide.

14. A system for detecting a concentration of carbon monoxide, the system comprising:
a membrane for passing of the carbon monoxide from outside of a wall transformer/power-supply to inside the wall transformer/power-supply, the membrane on a surface of the wall transformer/power-supply;
a sensor within the wall transformer/power-supply and coupled to the membrane for receiving the carbon monoxide, the sensor provides an electrical signal proportional to the concentration of the carbon monoxide;
a circuit within the wall transformer/power-supply, the circuit receives the electrical signal and compares the electrical signal to a concentration threshold and the circuit alerts if the electrical signal exceeds the concentration threshold;
a pair of prongs emanating from the wall transformer/power supply for inserting into a power outlet and making an electrical and mechanical connection to the power outlet;
a power conversion circuit accepts an electrical power from the pair of electrical contacts and the power conversion circuit converts the electrical power to a low-voltage provides power for providing operational and charging power to a device connected to the wall transformer/power-supply.

15. The system for detecting the concentration of the carbon monoxide of claim 14, wherein when the circuit alerts, a sound is emitted from a sound emitting device of the wall transformer/power-supply.

16. The system for detecting the concentration of the carbon monoxide of claim 14, wherein when the circuit alerts, a signal is sent from the circuit to the device connected to the wall transformer/power-supply by a wire and the device emits a sound.

17. The system for detecting the concentration of the carbon monoxide of claim 16, wherein when device is a cellphone.

18. The system for detecting the concentration of the carbon monoxide of claim 14, wherein when the circuit alerts, a signal is sent from the circuit to the device by a wireless transceiver and the device emits a sound.

19. The system for detecting the concentration of the carbon monoxide of claim 18, wherein the device is a cellphone.

20. The system for detecting the concentration of the carbon monoxide of claim 19, wherein when the circuit alerts, the cellphone transmits a message to another cellphone.
